(19) United States
(12) Patent Application Publication
(10) Pub. No.: US 2015/0248780 A1
(43) Pub. Date: Sep. 3, 2015

(54) Touchscreen Contouring Method for Radiation Oncology Treatment Planning and Treatment Administration

(71) Applicants: Carlos Daniel Caridad, Hawthorne, CA (US); Marcos Abraham Nassar, Los Angeles, CA (US)

(72) Inventors: Carlos Daniel Caridad, Hawthorne, CA (US); Marcos Abraham Nassar, Los Angeles, CA (US)

(21) Appl. No.: 13/866,011

(22) Filed: Apr. 18, 2013

(51) Int. Cl. G06T 11/60; G06T 5/00; G06T 7/00; A61N 5/10

(52) U.S. Cl. CPC G06T 11/60; A61N 5/1039; G06T 5/006; G06T 7/0079; G06T 2207/30096

(57) ABSTRACT

An improved method to draw the contour of the outline of a scanned tumor image for radiation oncology treatment planning and treatment administration using a touchscreen device and a stylus.

### CROSS REFERENCES TO RELATED APPLICATIONS

This application claims the benefit of PPA No. 61/689,036 filed Jul. 7, 2012, by Carlos Caridad, under the title: “Touch Screen Contouring Device”, one of the present inventors, which is incorporated by reference.

Related Applications This application claims the benefit under 35 U.S.C. §119(e) of U.S. Provisional Application Ser. No. 60/518,020, filed Oct. 7, 2003, titled “Planning System, Method, and Apparatus for Conformal Radiation Therapy,” which is incorporated by reference in its entirety.

### BACKGROUND OF THE INVENTION

1. Field of the Invention

The present invention relates generally to radiation therapy, and more specifically to conformal radiation therapy of tumors, and in particular to radiation therapy treatment planning methods, and apparatus for conformal radiation therapy.

2. Description of Related Art

One of the methods for treating cancerous tumors involves the use of radiation to destroy cancerous cells. The goal of conformal radiation therapy is to destroy cancerous cells while avoiding damage to the surrounding healthy tissue and organs. This radiation consists of a beam of high energy particles that is aimed at the cancerous tumor. In order to irradiate only the tumor and not the surrounding healthy tissue, the beam needs to be shaped to conform to the shape of the tumor. This is accomplished by a variable mechanical aperture through which the radiation beam passes, and which takes the shape of the outline of a scanned tumor image.

A conformal radiation therapy system includes the radiation source, the aperture, the hardware required to deliver the beam to the area being treated, a computer that controls the equipment, and specialized software used to define all the parameters that form part of the treatment plan, known as the Radiation Oncology Plan. Such parameters include the intensity of the beam, its duration, the shape of the aperture, and various other parameters related to the treatment. Systems such as this may include but are not limited to: IMRT (Intensity Modulated Radiation Therapy), 3 dimensional (3D) conformal Radiation Therapy, and IGRT (Image Guided Radiation Therapy).

The Radiation Oncology Plan, which is created by a radiation oncologist, defines the area to be treated by drawing a contour over the image of the scanned tumor on the computer screen. This contour will control the shape of the IMRT's aperture for presice delivery of the radiation beam.

One of the procedures currently used to create this drawing, or contour, consists of using the computer mouse to follow the outline of the scanned tumor on the screen. However, a computer mouse is not designed, nor suited to follow an outline accurately. One of the reasons is the error inherent in the feedback loop of eye-hand coordination. This error occurs because the mouse is being moved by the operator's hand on the table or mouse pad, while the eye follows the cursor on the screen and tries to guide it along the edge of the tumor's image. In other words, there is a visual disconnect between the hand controling the mouse and the cursor moving on the screen.

This challenge results in repetitive editing of the contouring line generated by the mouse movement, until the operator decides that the contour is close enough to the shape being outlined. This continuous editing is a time consuming process that is prone to errors and results in operator's physical and mental fatigue that lead to a contour that may not be as precise as desired. As a consequence, either some healthy tissue gets unnecessarily irradiated or some cancerous tissue does not receive the required radiation dosis.

Even though the radiation oncology doctor may not have the skills to follow accurately the cancer tumor contour with the mouse, he cannot delegate this task since the law (Radiation Regulatory agencies) requires that a Radiation Oncology medical doctor must perform the radiation therapy contouring treatment plan.

Another method of creating the contour is by using a specialized image recognition software that automatically creates a set of possible contours based on the scaned image. Then the oncologist needs to select and reposition the most accurate one, or needs to edit the shape of one of them to better fit the tumor's outline. This editing is again done with the use of a mouse, and therefore is subject to the same errors described above.

The aforementioned shortcomings of the current Radiation Oncology contouring method makes it clear that a more effective, accurate and user friendly solution is needed.

### SUMMARY OF THE INVENTION

This invention describes the use of a touchscreen device, such as a computer touchscreen monitor, and a hand held pointing instrument, such as a stylus, to contour the shape on an image of a scanned cancerous tumor. The method provides for a precise, easy to use and highly convenient way of contouring, tracing, mapping, or outlining a shape on an image as desired.

By contouring, tracing, or mapping of medical diagnostic images or scans we mean tracing the outline, margin, or extent of various pathological entities, including cancer, neoplasm and tumors, for the purpose of planning and administering radiation therapy.

Such images include but are not limited to medical diagnostic scan images, such as MRIs scans, PET scans, CT scans, PET-CT scans fusion, SPECT-CT scans fusion, MRI-CT scans fusion, ultrasound images, thermal images, X-Rays, nuclear medicine SPECT scans and Ultrasound-MRI fusion. The use of touchscreen devices have been proposed before to facilitate some of the tasks asociated with radiation oncology therapy. For example Patents No. EP1687064 A2, EP1551510 A1 use touchscreens to manipulate data and to create soft buttons. However none of these patents makes use of the touchscreen to create the actual contour of a scanned tumor image.

### DETAILED DESCRIPTION OF PREFERRED EMBODIMENT

The invention consists in the use of a touch screen device and a stylus to directly contour the outline of a cancerous tumor image on the touch screen. The trace thus created will be stored in memory for further procesing and subsecuent use as needed to create the Radiation Therapy Planning.

An operator will trace or outline the area of interest on the image to be contoured, using the input drawing/contouring device. That tracing will be stored and displayed on the sensitive screen for further processing or use as needed; e.g. it can be used for cancer tumor contouring for Radiation Therapy Planning.

The touch screen device can be an integral part of the conformal radiation therapy system, such as an Image Modulated Radiation Therapy system (IMRT), or it can be external to the system.

In the case of an external device, the tranfer of the contour to the IMRT system can be done wirelesly or by other means such as a USB connection or a removable memory stick. The use of a wireless digital tablet allows the doctor to perform the contouring operation away from the planning computer. The doctor will be able to perform this procedure from any place where a wireless connection is available. Suitable touch-sensitive devices include, but are not limited to, digital tablets, smart phones, computer touch-sensitive screens, laptops with a touch-sensitive screen, digital drawing boards and other touchscreen devices.

Suitable pointing input devices include, but are not limited to, styluses, pencil erasers, fingers and finger nails.

Detailed Description of Ilustrations

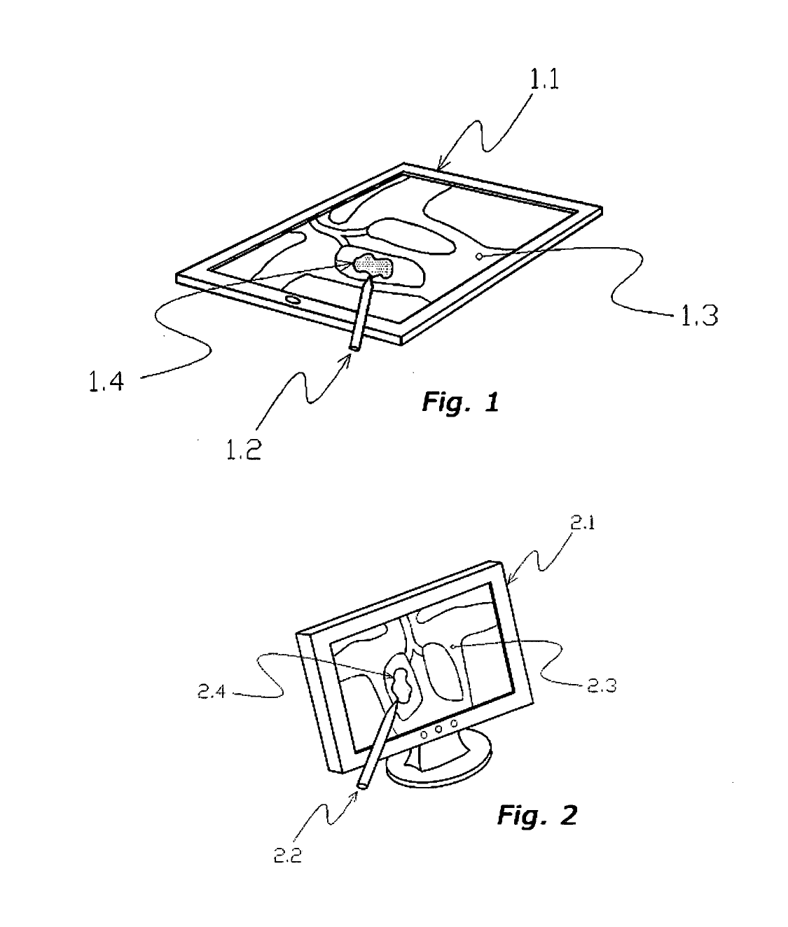

One embodiment of the invention is illustrated in FIG. 1. A digital tablet 1.1 is part of the input/output device of the invention. A stylus-pointing-drawing instrument 1.2 is another component of the invention's input. An image 1.3 of the scanned tumor to be contoured is displayed on the monitor 1.1. The touch-sensitive screen 1.1 records the movement of the stylus 1.2 that follows the outline of the scanned tumor image. The contour 1.4 of the scanned tumor image outline is thus created.

Another embodiment of the invention is illustrated in FIG. 2. A computer monitor with a touch-sensitive screen 2.1 is part of the input/output device of the invention. A stylus-pointing-drawing instrument 2.2 is another component of the invention's input. An image 2.3 of the scanned tumor to be contoured is displayed on the monitor 2.1. The touch-sensitive screen 2.1 records the movement of the stylus 2.2 that follows the outline of the scanned tumor image. The contour 2.4 of the scanned tumor image outline is thus created.

### Description and Operation of Alternative Embodiments

Another embodiment of the invention is to utilize a commercially available image recognition software that automatically creates a preliminary contour of the scanned tumor image's outline. This temporary outline needs to be edited to conform to a more precise contour of the scanned tumor image by using this invention.

This type of software automatically draws the outline of an image. Said temporary outline may not be very precise. The system operator, usually a radiation oncology medical doctor, must then edit the contour drawn by the image recognition software to better fit the outline of the scanned tumor according to the original radiation treatment plan. This editing operation is performed by using the stylus 1.2 on the touchscreen 1.1 as described in Operation of the Invention.

### How Do the Components Connect

1. Images that require contouring or tracing services are inputted or uploaded into the computer system to be displayed on the touch screen or 2D sensitive viewing device.

2. The drawing device (FIGS. 1.2 and 2.2) is used to contour or outline the area of interest, leaving the tracing on the Input/output screen device.

3. A wireless transmitter/receiver will send and receive data from and to any other computer device as needed.

4. Any visual information inputted on the device described on (1), can be stored and processed on a computer system nearby. This information can be transferred wirelessly or stored in a memory chip, and shared with any other computerized system.

5. All information inputted by means of the device described on (1) can be stored and transferred to another computer device or system, or other inputting devices such as described in (1), and/or similar devices or systems.

6. The touchscreen and stylus will interact with the contour automatically created by the image recognition software.

### Operation of Invention

This invention describes a new process of drawing the contour of a scanned tumor image. It replaces the current IMRT computer contouring planning system to shape the radiation beam of a radiation machine or linear accelerator, to treat a patient's cancerous tumor. This process uses a stylus to directly draw the contour of the scanned tumor image on a touch-sensitive screen.

Contour 1.4 is a line manually created around a tumor's edge as the stylus 1.2 touches the screen 1.1 and follows the outline of the tumor's image 1.3. This contour 1.4 of the scanned cancerous tumor image 1.3 is then used by the IMRT computer's contouring planning system to shape the radiation beam according to the radiation oncology treatment plan.

This invention eliminates the eye-hand coordination error and fatigue inherent in the use of a mouse to draw the contour on the scanned tumor image. The prior art relies on the computer mouse and screen cursor to follow the outline of the image that needs to be contoured. This new contouring process eliminates the use of the computer mouse and the reliance on a screen cursor.

The new process also eliminates the strain and fatigue caused on the operator's arm and hand when using a computer mouse to guide the cursor to create the contour, enhancing the precision of the outline.

### Advantages

1. Releases the radiation oncology doctor from the awkward, inconvenient and difficult-to-use computer mouse of prior art to create the tumor's contour.

2. Allows the doctor the freedom, convenience and comfort of being mobile, i.e. completing the contouring task from any location away from the planing computer.

3. Reduces the time required to create the contour of a scanned image.

4. Cancer patients benefit by having a more accurate delimitation of the tumor contour margins, thereby sparing adjacent normal healthy tissue from unnecessary radiation.

5. This also means the therapeutic radiation will be targeted more precisely to the cancerous tumor volume, i.e. is less likely to miss areas of the tumor's margins.

6. Eliminates the eye-hand coordination error and fatigue inherent in the use of a mouse to draw the contour on the scanned tumor image.

7. Eliminates the reliance on a screen cursor.

8. Eliminates the strain and fatigue caused on the operator's arm and hand when using a computer mouse to guide the cursor to create the contour, enhancing the precision of the outline.

### Conclusion

Thus the reader will see that at least one embodiment of the Touch Screen Contouring Method provides a more accurate, reliable, faster, fatigue-free and easy to use method of creating the contour of a scanned tumor image by using a digital tablet and a stylus instead of a computer mouse and a cursor on a computer screen. This Touch Screen Contouring Method saves time and money when compared with current contouring methods.

This invention also reduces the error associated with the use of a computer mouse and the eye-hand coordination required for the current method.

While the above description contains many specifics, these should not be construed as limitations on the scope of the invention, but rather as an exemplification of the two (manual and automatic contour generation) preferred embodiments thereof.

### BRIEF DESCRIPTION OF THE DRAWINGS

1.1 Digital Tablet

A touchscreen digital tablet that is part of the input/output device of the invention.

1.2 Stylus

A pointing-drawing instrument that is used as an input device.

1.3 Scanned Image

The image of the scanned tumor to be contoured is displayed on the digital tablet screen 1.1.

1.4 Contour

The hand-drawn contour of the scanned tumor image 1.3.

2.1 Touch-sensitive computer monitor

A computer monitor with a touch-sensitive screen that is part of the input/output device of the invention.

2.2 Stylus A pointing-drawing instrument that is used as an input device.

2.3 Scanned Image

The image of the scanned tumor to be contoured is displayed on the tablet screen 2.1.

2.4 Contour

The hand-drawn contour of the scanned tumor image 2,3.

1. A method for contouring the outline of a scanned image comprising a touchscreen device, a stylus and a communications network in communication with the treatment plan computer. The touchscreen device includes: a drawing software application (or app) and a memory to store data.

2. The method of claim 1 wherein said scanned image is that of a cancer tumor.

3. A method for contouring the outline of said scanned image by following said outline on a touchscreen device with said stylus.

4. A method of modifying, i.e. editing, a contour automatically generated by an image recognition software by following the correct outline of said scanned image using a stylus on a touchscreen device.

5. A method for contouring said outline of said image of a scanned tumor using said touchscreen device and a stylus wherein said touchscreen device is based on, but not limited to, a capacitive or a resistive technology.

6. A method for contouring the outline of said image where said touchscreen device is a digital tablet, such as, but not limited to, an iPad, an iTouch or a Microsoft Surface digital tablet.

7. The method of claim 1 wherein said touchscreen device is a smart phone.

8. The method of claim 1 wherein said touchscreen device is a touchscreen monitor of a laptop computer.

9. The method of claim 1 wherein said touchscreen device is a touchscreen monitor of a notebook computer.

10. The method of claim 1 wherein said touchscreen device is a touchscreen monitor of a desktop computer.

11. The method of claim 1 wherein said touchscreen device is a digital drawing board.

12. The method of claim 1 wherein said touchscreen device is an application-specific touchscreen monitor.

13. The method of claim 1 wherein said touchscreen device is a computer tablet, such as but not limited to a tablet PC or tablet Personal Computer.

14. The method of claim 1 wherein the stylus is replaced by a pointing object such as, but not limited to, a pencil eraser, a finger, or a finger nail.

15. The method of claim 1 applied to radiation therapies including but not limited to Intensity Modulated Radiation Therapy (IMRT), 3 dimensional (3D) Conformal Radiation Therapy, Image Guided Radiation Therapy (IGRT) and Dynamic Adaptive RadiEtion Therapy (DART™).

16. The method of claim 1 wherein the scanned image is that of, but not limited to, a tumor.

17. The method of claim 1 applied to medical applications.
